(19) United States
(12) Patent Application Publication
(10) Pub. No.: US 2007/0191856 A1
(43) Pub. Date: Aug. 16, 2007

(54) Adjustable height spinal distractor

(75) Inventors: Carlos E. Gil, Collierville, TN (US); Bret Matthew Wilfong, Southaven, MS (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/343,687

(22) Filed: Jan. 31, 2006

(51) Int. Cl. A61B 17/58

(52) U.S. Cl. 606/90

(57) ABSTRACT

A distractor assembly for preparing an intervertebral disc space between a first vertebra and a second vertebra is described. The distractor assembly comprises a cross bar and first and second distracting arms connected to the cross bar. Each of the distracting arms includes a base portion and a moveable portion connected to the base portion via a length adjustment assembly operable to cause translation of the moveable portion relative to the base portion, thereby to adjust a length of the distracting arm

### BACKGROUND

The distraction of adjacent vertebrae is a common requirement when treating trauma or disease to the spine. For example, reconstruction of a damaged joint with a functional joint prosthesis to provide motion and to reduce deterioration of the adjacent bone and adjacent joints is a desirable treatment option for many patients. For the surgeon performing the joint reconstruction, specialized instrumentation and surgical methods may be useful to facilitate precise placement of the prosthesis.

### SUMMARY

In one embodiment, a distractor assembly for preparing an intervertebral disc space between a first vertebra and a second vertebra comprises a cross bar and first and second distracting arms connected to the cross bar. Each of the distracting arms comprises a base portion and a moveable portion connected to the base portion via a length adjustment assembly operable to cause translation of the moveable portion relative to the base portion, thereby to adjust a length of the distracting arm.

In another embodiment, a method of using a distractor assembly to prepare an intervertebral disc space between a first vertebra and a second vertebra includes attaching first and second distractor arms of the distractor assembly to anchoring devices connected to the vertebrae and adjusting a length of one of the distractor arms using a length adjustment assembly connecting a base portion of the distractor arm to a moveable portion of the distractor arm.

### BRIEF DESCRIPTION OF THE DRAWINGS

FIG. 1 is a sagittal view of a vertebral column having a damaged disc.

FIG. 2 is an isometric view of a distractor assembly according to an embodiment of the current disclosure.

FIG. 3 is a cross-sectional view of a portion of the distractor assembly of FIG. 2 including a length adjustment mechanism thereof.

FIG. 4 is an isometric view of the distractor assembly of FIG. 2 showing an alternative positioning of the flexible shafts thereof.

FIG. 5 is an isometric view of attachment assembly portions of the distractor assembly of FIG. 2.

FIG. 6 is an isometric view of an anchoring device according to an embodiment of the current disclosure.

FIG. 7 is a partial environmental view of the distractor assembly of FIG. 2 coupled with the anchoring device of FIG. 6.

FIG. 8 is an isometric view of a distractor assembly according to an alternative embodiment of the current disclosure.

### DETAILED DESCRIPTION

The present disclosure relates generally to the field of orthopedic surgery, and more particularly to a distraction device for use during vertebral reconstruction using an intervertebral prosthesis. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the invention relates.

Referring first to FIG. 1, the numeral 10 refers to a human anatomy having a joint location which in this example includes an injured, diseased, or otherwise damaged intervertebral disc 12 extending between vertebrae 14, 16. The damaged disc may be replaced by an intervertebral disc prosthesis 18 which may be a variety of devices including the prostheses that have been described in U.S. Pat. Nos. 5,674,296; 5,865,846; 6,156,067; 6,001,130 and in U.S. Patent Application Nos. 2002/0035400; 2002/0128715; and 2003/0135277, which are incorporated by reference herein.

In other embodiments, various different reasons may exist for accessing the intervertebral disc 12, or the disc space. These include such procedures as inserting one or more fusion implants, injecting material into the disc space, examining the disc space treated in a prior procedure, and so forth.

Continuing with the prior example, during a surgical technique for repairing the damaged disc 12, all or a portion of the disc may be excised. This procedure may be performed using an anterior, anterolateral, lateral, or other approach known to one skilled in the art, however, the following embodiments will be directed toward a generally anterior approach. Generally, the tissue removal procedure may include positioning and stabilizing the patient. Fluoroscopic or other imaging methods may be used to assist with vertebral alignment and surgical guidance. Imaging techniques may also be used to determine the proper sizing of the intervertebral prosthesis 18. In one embodiment, a sizing template may be used to pre-operatively determine the correct prosthesis size. The tissue surrounding the disc space may be retracted to access and verify the target disc space. The area of the target disc may be prepared by removing excess bone, including osteophytes which may have developed, and other tissues which may include portions of the annulus and all or portions of the nucleus pulpous. The tissue removal procedure, which may include a discectomy procedure, may alternatively or additionally be performed after alignment and/or measurement procedures have been taken.

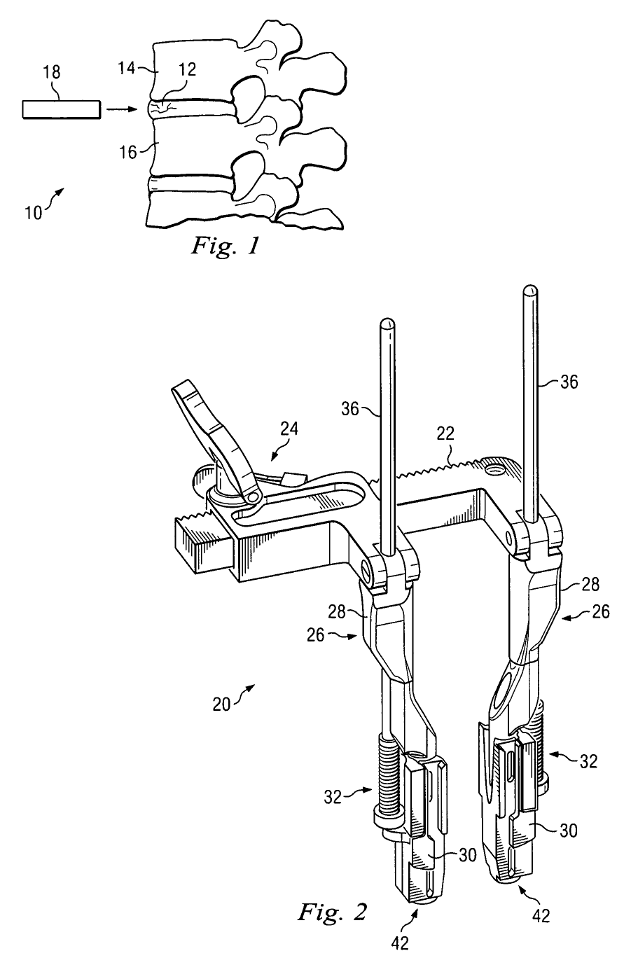

After the tissue is removed, various alignment procedures may be conducted to align the intervertebral space in preparation for the disc prosthesis 18. These alignment procedures may include introduction of a distractor assembly, such as that shown in FIG. 2 and designated by a reference numeral 20. The distractor assembly 20 may include a cross bar member 22 having a securing mechanism 24. A pair of distracting arms 26 may be attached to the cross bar member 22. A variety of securing mechanisms 24 may be used to maintain a selected distance between the distracting arms 26, including, for example, a ratchet system, clamps, threaded connectors, pins, gripping hardware, or other fasteners. At least one of the distracting arms 26 may be movably connected to the cross bar member 22 with the securing mechanism 24.

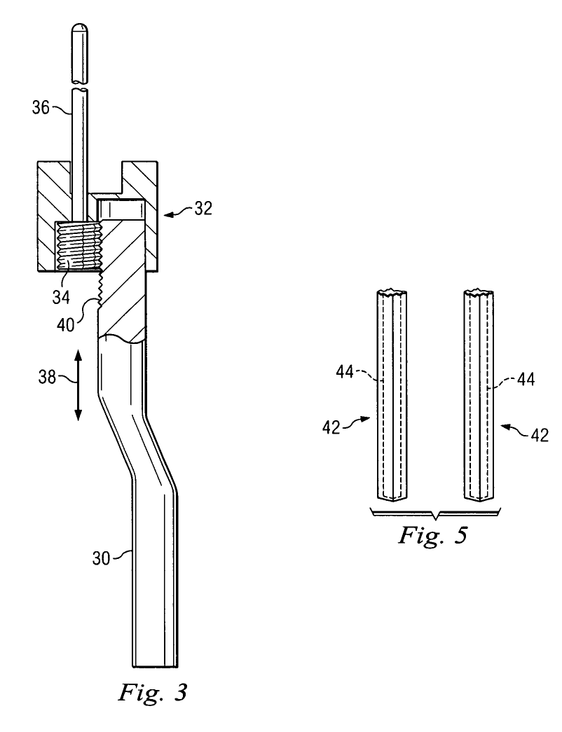

Each of the distracting arms 26 includes an arm base 28 and a moveable arm portion 30 moveably connected to the arm base by a length adjustment mechanism 32 for maintaining a selected length of the distracting arm, and thereby a selected height of the distractor assembly 20, as will be described. In one embodiment, as best illustrated in FIG. 3, each of the length adjustment mechanisms 32 includes a helical screw 34 connected to a shaft 36 such that rotation of the shaft 36 results in a corresponding rotation of the helical screw about an axis 38 comprising an axis of rotation of the screw passing through the center of the shaft and screw. When using an anterior surgical technique, the axis 38 may be an anterior-posterior axis.

Continuing with reference to FIG. 3, each of the moveable arm portions 30 includes a threaded section 40 for engaging threads of the helical screw 34 such that rotation of the screw using the shaft 36, as described above, results in translation of the moveable arm portion relative to the arm base 28 in a direction parallel to the axis 38. Rotation of the helical screw 34 in one direction will result in a lengthening of the distracting arm 46, while rotation of the helical screw in the opposite direction will result in a shortening of the distracting arm. It will be recognized that in this manner the length of each of the distracting arms 26 can be adjusted independently of that of the other distracting arm.

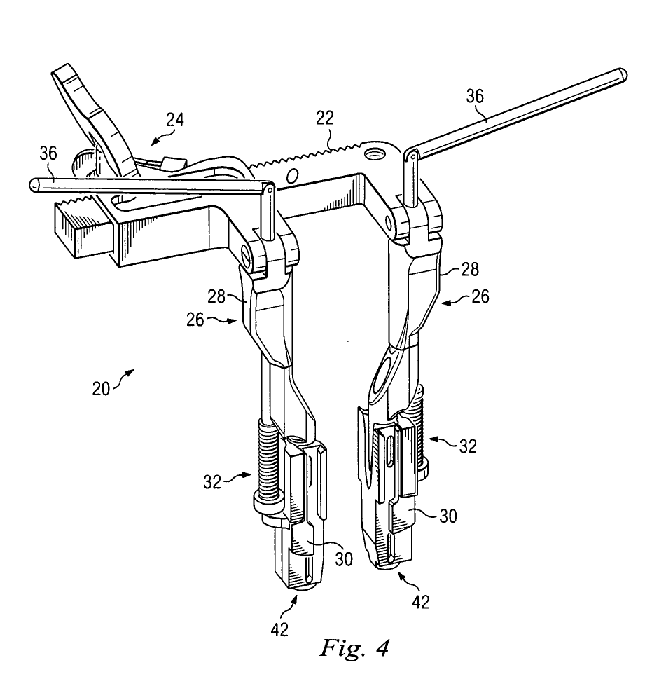

As illustrated in FIG. 4, in some embodiments, the shaft 36 may be flexible, such that once the length of one of the distracting arms 26 has been adjusted as desired, the flexible shaft 36 thereof may be bent such that it remains out of the way during the remainder of the surgical technique.

In an alternative embodiment, the shaft 36 may be partially or totally removable from the distracting arms. For example, the shaft 36 may have an engaging mechanism such as a square, hex, or Phillips drive that selectively connects to the helical screw 34. In these embodiments, the shaft 36 may not be flexible. In yet another embodiment, a first portion of the shaft 36 may extend just beyond the arm base 28. A second portion of the shaft 36 may then be selectively coupled to the first portion when a change in the amount of distraction is to be performed, and then removed from the first portion during other procedures.

Referring again to FIG. 2, each of the distracting arms 26 may include an attachment mechanism 42. In the embodiment illustrated in FIG. 2 and as shown in greater detail in FIG. 5, the attachment mechanisms 42 may include hollow cavities 44. In some embodiments, as shown, the distracting arms 26 may have relatively flat end portions, but in alternative embodiments, the end portions may be angled or curved. The attachment mechanisms 42 may be used to locate, hold, and/or guide anchoring devices as will be described below and may include stops or other features useful for position verification or instrument support. A variety of other embodiments of attachment mechanisms may be provided, including, for example, attachment mechanisms including pins and hollow recesses and wherein one or more of the walls of the hollow recesses may have elongated openings.

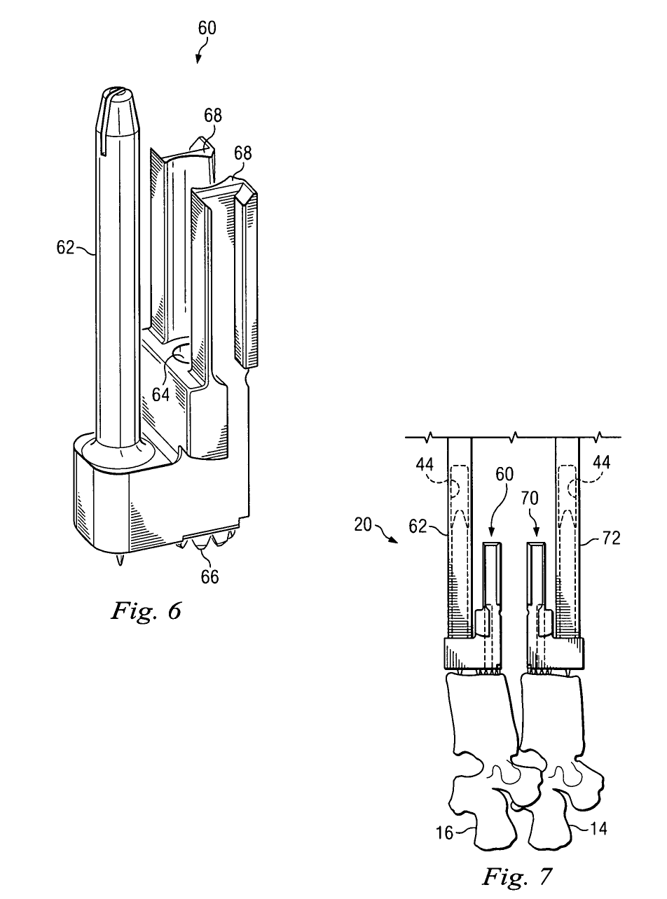

Referring now to FIGS. 6 and 7, an anchoring device 60 may include a connecting portion 62, a vertebral body attachment portion 64, a seat 66, and constraint portions 68. The anchoring device 60 may attach to one of the distracting arms 26 by inserting the connecting portion 62 into one of the hollow cavities 44. An opposing anchoring device 70 having the same or similar features anchoring device 60 including an attachment portion 72 may be attached to the other of the distracting arms 26.

The anchoring devices 60, 70 may be of a configuration which attaches to the vertebral bodies 14, 16 and permits independent movement of the vertebral bodies 14, 16 in the sagittal plane while maintaining alignment of the vertebral bodies 14, 16 in the transverse and coronal planes.

A variety of alternative anchoring devices with alternative means for attaching to a distractor assembly may be selected which permit at least some movement of the vertebral bodies 14, 16 in a single plane, such as a sagittal plane. In some embodiments, the connection between the distractor assembly and the anchoring devices may be selectably fixed, pivotable, or movable in a linear direction.

It will be recognized that, although particular embodiments of attachment mechanisms 42 and corresponding anchoring devices are described herein, it is anticipated that there will be will be any number of different types of attachment mechanisms and corresponding anchoring devices that may be practiced with the invention. Moreover, the detailed description herein of particular embodiments of such mechanisms and corresponding devices does not evince an intent to limit the practice of the invention to only to those embodiments so described.

With the vertebral bodies 14, 16 distracted and the anchoring devices 60, 70 attached to the vertebral bodies 14, 16, various measurements, such as a depth measurement, may be performed at the disc site to determine the proper sizing of instrumentation and devices to be used throughout the remainder of the surgical technique. Next, further preparation of the vertebral endplate surfaces is performed using a cutting instrument, such as a burr or other cutting surface known in the art. The cutting instrument may also include a telescoping shaft to permit lengthening of the cutting instrument. After the vertebral endplates are prepared, the intervertebral prosthesis 18 may be inserted into the prepared space using any of a variety of insertion methods. After the prosthesis 18 is implanted, the tension on the distractor assembly 20 may be released and the distractor assembly 20 removed. With all instrumentation removed from the disc site, the wound may be closed.

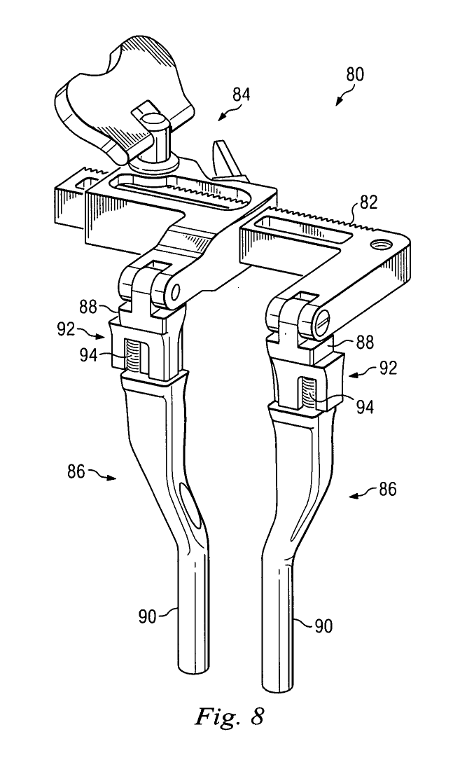

Referring now to FIG. 8, another embodiment of a distractor assembly, designated by a reference numeral 80, may include a cross bar member 82 having a securing mechanism 84. A pair of distracting arms 86 may be attached to the cross bar member 82. As with the distractor assembly 20, a variety of securing mechanisms 84 may be used to maintain a selected distance between the distracting arms 86 and at least one of the distracting arms 86 may be movably connected to cross bar member 82 with the securing mechanism 84.

Each of the distracting arms 86 includes an arm base 88 and a moveable arm portion 90 moveably connected to the arm base by a length adjustment mechanism 92 for maintaining a selected length of the distracting arm, as will be described. In the embodiment illustrated in FIG. 8, the length adjustment mechanism 92 comprises a helical screw 94, at least a portion of which is exposed such that it may be rotated by a user using his thumb or other finger. As with the distractor assembly 20 shown in FIG. 2, each of the moveable arm portions 90 includes a threaded section (not shown in FIG. 8), similar to the threaded section 40 (FIG. 2) for engaging threads of the helical screw 94 such that rotation of the screw in the manner described above results in translation of the moveable arm portion relative to the arm base 88 in a direction parallel to an axis of rotation through the center of the screw 94. It will be recognized that in this manner, the length of each of the distracting arms 86 can be adjusted independently of that of the other distracting arm.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims. In the claims, means-plus-function clauses are intended to cover the structures described herein as performing the recited function and not only structural equivalents, but also equivalent structures.

What is claimed is:

1. A distractor assembly for preparing an intervertebral disc space between a first vertebra and a second vertebra, the distractor assembly comprising:
a cross bar;
first and second distracting arms connected to the cross bar;
wherein each of the distracting arms comprises a base portion and a moveable portion connected to the base portion via a length adjustment assembly operable to cause translation of the moveable portion relative to the base portion, thereby to adjust a length of the distracting arm.

2. The distractor assembly of claim 1 wherein at least one of the distracting arms is moveably connected to the cross bar via a securing mechanism.

3. The distractor assembly of claim 1 wherein the length adjustment assembly comprises a helical screw.

4. The distractor assembly of claim 3 wherein moveable portion comprises a threaded section for engaging threads of the helical screw such that rotation of the helical screw results in translation of the moveable portion.

5. The distractor assembly of claim 4 wherein translation of the moveable portion relative to the base portion occurs in a direction parallel to an axis of rotation of the helical screw.

6. The distractor assembly of claim 4 further comprising a shaft attached to the helical screw for rotating the helical screw about an axis of rotation thereof.

7. The distractor assembly of claim 6 wherein the shaft is flexible.

8. The distractor assembly of claim 4 wherein at least a portion of the helical screw is exposed such that the helical screw may be manually rotated by applying force to the exposed portion.

9. A method of using a distractor assembly to prepare an intervertebral disc space between a first vertebra and a second vertebra;
attaching first and second distractor arms of the distractor assembly to anchoring devices connected to the vertebrae; and
adjusting a length of one of the distractor arms using a length adjustment assembly connecting a base portion of the distractor arm to a moveable portion of the distractor arm.

10. The method of claim 9 wherein the length adjustment assembly comprises a helical screw and wherein the moveable portion comprises a threaded section for engaging threads of the helical screw and wherein the adjusting a length of one of the distractor arms comprises rotating the helical screw to cause translation of the moveable portion relative to the base portion.

11. The method of claim 10 wherein the rotating the helical screw comprises rotating a shaft attached to the helical screw such that rotation of the shaft results in rotation of the helical screw.

12. The method of claim 10 further comprising bending the shaft upon completion of the adjusting.

13. The method of claim 10 wherein the rotating the helical screw comprises applying rotational force directly to an exposed portion of the helical screw.

14. A distractor assembly for preparing an intervertebral disc space between a first vertebra and a second vertebra, the distractor assembly comprising:
a cross bar;
first and second distracting arms connected to the cross bar, wherein each of the distracting arms comprises:
a base portion;
a moveable portion comprising a threaded section; and
a length adjustment assembly for connecting the moveable portion to the base portion, the length adjustment assembly comprising a helical screw.

15. The distractor assembly of claim 14 wherein at least one of the distracting arms is moveably connected to the cross bar via a securing mechanism.

16. The distractor assembly of claim 14 wherein the helical screw includes threads for engaging the threaded section of the moveable portion such that rotation of the helical screw results in translation of the moveable portion relative to the base portion.

17. The distractor assembly of claim 16 wherein translation of the moveable portion relative to the base portion occurs in a direction parallel to an axis of rotation of the helical screw.

18. The distractor assembly of claim 16 further comprising a shaft attached to the helical screw for rotating the helical screw about an axis of rotation thereof.

19. The distractor assembly of claim 18 wherein the shaft is flexible.

20. The distractor assembly of claim 16 wherein at least a portion of the helical screw is exposed such that the helical screw may be manually rotated by applying force to the exposed portion.
